(19) United States
(12) Patent Application Publication
(10) Pub. No.: US 2012/0109200 A1
(43) Pub. Date: May 3, 2012

(54) METHODS AND DEVICES FOR RESTRICTING FLEXION AND EXTENSION OF A SPINAL SEGMENT

(75) Inventors: Colin Cahill, Portola Valley, CA (US); Manish Kothari, San Rafael, CA (US); Todd Alamin, Woodside, CA (US); Louis Fielding, San Carlos, CA (US)

(73) Assignee: SIMPIRICA Spine, Inc., San Carlos, CA (US)

(21) Appl. No.: 13/095,604

(22) Filed: Apr. 27, 2011

Related U.S. Application Data
(63) Continuation of application No. PCT/US2009/065658, filed on Nov. 24, 2009.
(60) Provisional application No. 61/117,428, filed on Nov. 24, 2008.

(51) Int. Cl. A61B 17/70; A61B 17/88

(52) U.S. Cl. 606/248; 606/279

(57) ABSTRACT

Methods and devices for restricting movement of a spinal segment by providing an adjustable constraining device that includes a tether and a compliance member coupled together. The tether is coupled to a superior spinous process and an inferior spinous process or a sacrum so that the construct of the compliance member and tethers provides a force resistant to flexion and a force resistant to extension of a spinal segment. In some embodiments, the construct of the compliance member and tethers may provide only a force resistant to extension.

### CROSS-REFERENCE TO RELATED APPLICATIONS

This application is a continuation of International Patent Application No. PCT/US2009/065658 (Attorney Docket No. 026398-001110PC) filed Nov. 24, 2009, which claims the benefit of U.S. Provisional No. 61/117,428 (Attorney Docket No. 026398-001100US) filed Nov. 24, 2008, the contents of which are incorporated herein by reference.

### BACKGROUND OF THE INVENTION

1. Field of the Invention

The present invention generally relates to medical methods and devices. More particularly, the present invention relates to methods and devices used to restrict flexion and/or extension of a spinal segment. This includes but is not limited to treatment of patients having back pain, spinal abnormalities or other conditions of the spine and/or vertebral column.

Spinal stability is highly dependent on the patency of attached soft tissue such as ligaments, spinal load and posture as well as task requirements. In particular, the ligaments and disc play a key role in keeping each spine segment stable and aligned. Degeneration of ligaments, disc or other tissue structures can lead to inability of the spinal segment to maintain stability even over a normal range of loads. Instability of the lumbar spine has been suggested to be both a cause and a consequence of acute, recurring or chronic low back pain. It is estimated that 80% of the general population will suffer from backache or lumbago during their lifetime (Fryomoyer et al., “An Overview of the Incidence and Costs of Low Back Pain” Orthrop. Clin. North Am. (1991) 22:263-271).

A major source of chronic low back pain is discogenic pain, also known as internal disc disruption. Patients suffering from discogenic pain tend to be young, otherwise healthy individuals who present with pain localized to the back. Discogenic pain usually occurs at the discs located at the L4-L5 or L5-S1 junctions of the spine. Pain tends to be exacerbated when patients put their lumbar spines into flexion (i.e. by sitting or bending forward) and relieved when they put their lumbar spines into extension (i.e. by standing or arching backwards). Flexion and extension are known to change the mechanical loading pattern of a lumbar segment. When the segment is in extension, the axial loads borne by the segment are shared by the disc and facet joints (approximately 30% of the load is borne by the facet joints). In flexion, the segmental load is borne almost entirely by the disc. Furthermore, the nucleus shifts in the posterior direction, changing the loads on the posterior portion of the annulus (which is innervated), likely causing its fibers to be subject to tension and shear forces. Segmental flexion, then, increases both the loads borne by the disc and causes them to be borne in a more painful way. Discogenic pain can be quite disabling, and for some patients, can dramatically affect their ability to work and otherwise enjoy their lives.

Current treatment alternatives for patients diagnosed with chronic back pain are quite limited. Many patients follow a conservative treatment path, such as physical therapy, massage, anti-inflammatory and analgesic medications, muscle relaxants, and epidural steroid injections, but typically continue to suffer with a significant degree of pain. Other patients elect to undergo spinal fusion surgery, which commonly requires discectomy (removal of the disk) together with fusion of adjacent vertebra. Fusion may or may not also include instrumentation of the affected spinal segment including, for example, pedicle screws and stabilization rods. Fusion is not lightly recommended for discogenic pain because it is irreversible, costly, associated with high morbidity, and has questionable effectiveness. Despite its drawbacks, however, spinal fusion for discogenic pain remains common due to the lack of viable alternatives.

One method, that is not commonly used in practice, but has been approved for use by the United States Food and Drug Administration (FDA), is the application of bone cerclage devices which can encircle the spinous processes or other vertebral elements and thereby create a restraint to motion. Physicians typically apply a tension or elongation to the devices that applies a constant and high force on the anatomy, thereby fixing the segment in one position and allowing effectively no motion. These devices are designed for static applications and are not designed to allow for dynamic elastic resistance to flexion across a range of motion. The purpose of bone cerclage devices and other techniques described above is to almost completely restrict measurable motion of the vertebral segment of interest. This loss of motion at a given segment gives rise to abnormal loading and motion at adjacent segments, which can lead eventually to adjacent segment morbidity and other problems.

An alternative solution that avoids some of the challenges associated with cerclage devices involves the use of an elastic structure, such as tether structures, coupled to the spinal segment. The elastic structure can relieve pain by increasing passive resistance to flexion while often allowing substantially unrestricted spinal extension.

Such a spinal implant is illustrated in FIG. 2 of the present application and is disclosed in greater detail in U.S. Patent Publication No. 2005/02161017. The implant has been designed to inhibit spinal flexion while allowing substantially unrestricted spinal extension. The implant is placed over one or more adjacent pairs of spinous processes and provides an elastic restraint to the spreading apart of the spinous processes which occurs during flexion.

As illustrated in FIG. 2, an implant 10 as described in the 2005/02161017 publication typically comprises an upper strap component 12 and a lower strap component 14 joined by a pair of compliance members 16. The upper strap 12 is shown disposed over the top of the spinous process SP4 of L4 while the lower strap 14 is shown extending across the bottom of the spinous process SP5 of L5. The compliance member 16 will typically include an internal element, such as elastomeric members 72a and 72b (FIG. 7 in the 2005/02161017 publication) which are attached to inelastic cables 76a and 76b in such a way that the cables may be “elastically” or “compliantly” pulled apart as the spinous processes SP4 and SP5 move apart during flexion. In particular, the compliance or elasticity is provided by the cables compressing the elastomeric members 72a and 72b between stopper elements 78a, 78b, 80a, and 80b at their respective ends. In this way, the implant provides an elastic tension on the spinous processes which provides a force that resists flexion. The force increases as the processes move further apart and the rubber or elastomeric blocks become more compressed. Usually, the straps or cables themselves will be essentially non-compliant so that the degree of elasticity or compliance may be controlled and provided solely by the nature of the elastomeric members in compliance members 16.

While these devices are promising, some patients, such as those with a combination of back and leg pain, may benefit from a device that not only provides resistance to flexion but that also provides resistance to extension as well. Accordingly, the methods and devices described herein should also be useful for other spinal disorders or treatments associated with segmental extension, for which the prevention or control of spinal segmental extension is desired.

Other implants do provide some resistance to extension of the spinal segment. For example, one commercially available device uses a spacer to resist extension. To implant the device, the supraspinous ligament is temporarily lifted and displaced. The interspinous ligament between the two adjacent vertebrae of interest is then permanently removed and the spacer is inserted in the interspinous interspace. A tether is then wrapped around the processes of the two adjacent vertebrae, through adjacent interspinous ligaments, and then mechanically secured in place by the spacer or also by a separate component fastened to the spacer. The supraspinous ligament is then restored back to its original position. Such implants and accompanying surgical methods are not without disadvantages. These implants may subject the spinous processes to frequent, high loads during everyday activities, sometimes causing the spinous processes to break or erode. Furthermore, the spacer may put a patient into segmental kyphosis, potentially leading to long-term clinical problems associated with lack of sagittal balance. The process of securing the tethers is often a very complicated maneuver for a surgeon to perform, making the surgery much more invasive. And, as previously mentioned, the removal of the interspinous ligament is permanent. As such, the application of the device is not reversible. In addition, adjustment of the size of these implants in situ is often not possible. Thus, there still is a need to provide devices that not only provide resistance to flexion and extension of a spinal segment, but also that are less invasive and easier to implant.

Some examples of commercial devices that treat lumbar spinal stenosis by specifically and soley limiting symptomatic extension include the Superion™ Interspinous Spacer (VertiFlex®), Aperius™ and X-Stop® (Medtronic®) and the BacJac™ (Pioneer Surgical®). In addition to limiting extension only, the Coflex™ device (Paradigm Spine®) is sometimes pinned through the spinous processes to restrict both flexion and extension. These devices each suffer from one or more of at least the following shortcomings. Implantation can be traumatic and invasive. For example, nearby anatomy, including spinous processes can be damaged during placement or eroded over time. It can also be difficult or impossible to adjust the degree of extension limitation. Furthermore, most of these devices provide only a hard extension stop which can increase the impact of loads transmitted to the spinous processes, potentially leading to tissue damage and/or device settling. Thus, there is still a need for devices that overcome the previous disadvantages of current implants and further enable dynamic control of extension or both flexion and extension.

More recently, less invasive spinal implants have been introduced. These spinal implants are placed over one or more pairs of spinous processes and provide an elastic restraint to the spreading apart of the spinous processes occurring during flexion. Implantation does not require permanent removal of the interspinous ligaments and thus the device may be more easily explanted. The implants typically include a tether structure and a securing mechanism for the tether. The tether may be made from a flexible polymeric textile such as woven polyester (PET) or polyethylene (e.g. ultra high molecular weight polyethylene, UHMWPE); multi-strand cable, or other flexible structure. The tether is wrapped around the processes of adjacent vertebrae and then secured by the securing mechanism. The securing mechanism may involve the indexing of the tether and the strap, e.g., the tether and the securing mechanism include discrete interfaces such as teeth, hooks, loops, clamps, etc. which interlock the two. Many known implementations clamp a tether with the tip of a set-screw, or the threaded portion of a fastener. Other known methods use a screw or bolt to draw other components together to generate a clamping force. Other locking methods include the use of a friction fit. While these devices are promising, they still do not provide resistance to extension of the spinal segment and thus there is still a need for improved devices for patients with certain pathologies.

For at least these reasons, it is desirable to provide methods and devices that provide motion control to a spinal segment such as resistance to flexion and extension of the spinal segment. The methods and devices should be anatomically compatible, safe, effective, minimally invasive and easily implantable. As such, the following disclosure relates to methods and devices providing force resistance to extension and flexion of a spinal segment.

2. Description of the Background Art

Patents and published applications of interest include: U.S. Pat. Nos. 3,648,691; 4,643,178; 4,743,260; 4,966,600; 5,011,494; 5,092,866; 5,116,340; 5,180,393; 5,282,863; 5,395,374; 5,415,658; 5,415,661; 5,449,361; 5,456,722; 5,462,542; 5,496,318; 5,540,698; 5,562,737; 5,609,634; 5,628,756; 5,645,599; 5,725,582; 5,902,305; Re. 36,221; U.S. Pat. Nos. 5,928,232; 5,935,133; 5,964,769; 5,989,256; 6,053,921; 6,248,106; 6,312,431; 6,364,883; 6,378,289; 6,391,030; 6,468,309; 6,436,099; 6,451,019; 6,582,433; 6,605,091; 6,626,944; 6,629,975; 6,652,527; 6,652,585; 6,656,185; 6,669,729; 6,682,533; 6,689,140; 6,712,819; 6,689,168; 6,695,852; 6,716,245; 6,761,720; 6,835,205; 7,029,475; 7,163,558; Published U.S. Patent Application Nos. US 2002/0151978; US 2004/0024458; US 2004/0106995; US 2004/0116927; US 2004/0117017; US 2004/0127989; US 2004/0172132; US 2004/0243239; US 2005/0033435; US 2005/0049708; 2005/0192581; 2005/0216017; US 2006/0069447; US 2006/0136060; US 2006/0240533; US 2007/0213829; US 2007/0233096; US 2007/0239159; US 2008/0009866; US 2008/0108993; Published PCT Application Nos. WO 01/28442 A1; WO 02/03882 A2; WO 02/051326 A1; WO 02/071960 A1; WO 03/045262 A1; WO 2004/052246 A1; WO 2004/073532 A1; WO 2008/051806; WO 2008/051423; WO 2008/051801; WO 2008/051802; and Published Foreign Application Nos. EP 0322334 A1; and FR 2681525 A1. The mechanical properties of flexible constraints applied to spinal segments are described in Papp et al. (1997) Spine 22: 151-155; Dickman et al. (1997) Spine 22:596-604; and Garner et al. (2002) Eur. Spine J. S186-S191; Al Baz et al. (1995) Spine 20, No. 11, 1241-1244; Heller, (1997) Arch. Orthopedic and Trauma Surgery, 117, No. 1-2:96-99; Leahy et al. (2000) Proc. Inst. Mech. Eng. Part H: J. Eng. Med. 214, No.5: 489-495; Minns et al., (1997) Spine 22 No. 16:1819-1825; Miyasaka et al. (2000) Spine 25, No.6: 732-737; Shepherd et al. (2000) Spine 25, No.3: 319-323; Shepherd (2001) Medical Eng. Phys. 23, No.2: 135-141; and Voydeville et al. (1992) Orthop Traumatol 2:259-264. However, each of these references suffers from one or more of the disadvantages previously described.

In addition, the following applications may be of interest: U.S. patent application Ser. No. 11/076,469 (Attorney Docket No. 026398-000210US), filed on Mar. 9, 2005, which claims the benefit of prior U.S. provisional application No. 60/551,235, filed on Mar. 9, 2004; U.S. application Ser. No. 11/777,366 (Attorney Docket No. 026398-000110US); filed on Jul. 13, 2007; U.S. application Ser. No. 11/827,980 (Attorney Docket No. 026398-000120US); filed on Jul. 13, 2007; PCT application no. PCT/US2007/081815 (Attorney Docket No. 026398-000130PC), filed on Oct. 18, 2007; PCT application no. PCT/US2007/081822 (Attorney Docket No. 026398-000140PC), filed on Oct. 18, 2007; U.S. application Ser. No. 11/975,674 (Attorney Docket No. 026398-000150US), filed on Oct. 19, 2007; U.S. provisional application No. 61/059,530 (Attorney Docket No. 026398-000600US), filed on Jun. 6, 2008; U.S. application Ser. No. 12/106,103 (Attorney Docket No. 026398-000410US), filed on Apr. 18, 2008; and U.S. application Ser. No. 12/106,049 (Attorney Docket No. 026398-000151US), filed on Apr. 18, 2008.

### BRIEF SUMMARY OF THE INVENTION

The present invention provides methods and devices for controlling movement to treat back pain, spinal abnormalities or other conditions of the spine and/or vertebral column where a physician desires to provide force resistant to extension and flexion of a spinal segment of a patient. Such patient constraints may capture vertebral anatomy including, but not limited to, any combination of spinous processes, transverse processes, lamina and/or pedicles.

In a first aspect of the present invention, a device for controlling the movement of a spinal segment comprises a constraint coupled to a superior spinous process and an inferior spinous process. The constraint comprises a first strap configured to capture the superior surface of the superior spinous process and a second strap configured to capture the inferior surface of the inferior spinous process. A third strap is coupled to the first strap and is configured to capture the inferior surface of the superior spinous process. A fourth strap is coupled to the second strap and is configured to capture the superior surface of the inferior spinous process. One end of a first compliance member is connected to the first and third straps. The second end of the compliance member is connected to the second and fourth straps. The construct of the compliance member and straps provides resistance to extension and flexion of the spinal segment.

The straps on the inside of the interspinous space prevent the spinous processes from approximating. The straps could be constructed out of any material, including woven or unwoven fabric, that is substantially inextensible. Suitable materials include, but are not limited to, polyester, PET, UHMWPE, polyethylene or PEEK, for example. They could take the form of a leash, harness, lead, braid, plait, cord, belt, weave, strip, tie or band, for example. This provides advantages over a solid block, cushion or spacer implanted between the spinous processes because the straps: 1) increase flexibility; 2) minimize risk of damage to spinous processes by conforming to the bone surface; 3) cannot be easily dislodged; 4) are compatible with a variety of compliance members allowing for modular elasticity or resistance to extension; 5) deploy with less tissue removal and disruption than would be required to place a solid interspinous element between the same pair of spinous processes; 6) enable tunable adjustment of extension restriction to the needs of the patient; and 7) in combination with compliance elements, minimize peak impact loads on the spinous processes.

If desired, an additional compliance member can be added to the device described above. In this case, the second compliance member is located across the midline of the spinal segment. The second compliance member has a first end and a second end opposite the first end. One end of a second compliance member is connected to the first and third straps. The second end is connected to the second and fourth straps. The second compliance member may take the same form and provide similar resistant forces as the first compliance member or it may be different. The second compliance member is positioned essentially parallel to the first compliance member. The compliance member may comprise a spring or elastomeric member. Each of the straps may take the form of a leash, harness, lead, braid, plait, cord, belt, weave, strip, tie or band. They may be made of fabric. The device has a mechanism that adjusts to allow tightening around the spinous process to provide elastic resistance to extension and flexion of a spinal segment. The natural anatomy of the interspinous ligament remains substantially intact after implantation of the device.

In a second aspect of the present invention, a device for controlling the movement of a spinal segment comprises a first strap having a first end and a second end, wherein the first strap captures a superior spinous process. A second strap has first and second ends, and the second strap captures an inferior spinous process. The first and second straps are substantially inextensible. A first compliance member has a first end connected to at least one of the first and second ends of the first strap and a second end (opposite the first end) connected to at least one of the first and second ends of the second strap. The construct of the first compliance member and straps provides resistance to extension and flexion of the spinal segment.

An additional compliance member can be added to the device described above. In this case, the second compliance member has a first end and a second end opposite the first end. The second compliance member may be positioned across a spinal segment midline from the first compliance member. The first strap is coupled with the first end of the second compliance member and the second strap is coupled with the second end of the second compliance member. The device has a mechanism that adjusts to allow tightening around the spinous process. The natural anatomy of the interspinous ligament remains substantially intact after implantation of the device.

In still another aspect of the present invention, a method for restricting movement of a spinal segment comprises implanting a constraint. The constraint has a first adjustable strap and second adjustable strap with each strap coupled to at least one compliance member. A superior spinous process is captured with the first adjustable strap and an inferior spinous process is captured with the second adjustable strap. The straps are adjustable so that the compliance member and straps provide a force resistant to extension and flexion of the spinal segment. Adjusting the length or tension on the straps can set a relative distance or angle between the upper and lower spinous processes to a target value. Length and tension adjustment details can be found in U.S. application No. 61/093,922 (Attorney Docket No. 026398-000900US), filed on Sep. 3, 2008, the full disclosure incorporated herein by reference.

Additionally, capturing the spinous processes with the first and second adjustable straps does not require removal of the interspinous ligament. The first adjustable strap forms a first loop with a superior and an inferior segment that captures the superior spinous process. A second adjustable strap forms a second loop with a superior and an inferior segment that captures the inferior spinous process. Capturing may comprise encircling the superior spinous process with the first strap and encircling the inferior spinous process with the second strap. Adjusting the straps comprises adjusting the length or tension of the straps.

In yet another aspect of the present invention, a device for restricting movement of a spinal segment comprises a constraint having a superior loop and an inferior loop. The superior loop captures a superior spinous process and the inferior loop captures an inferior spinous process. A first compliance member has first and second ends which are opposite one another. The first end is connected to the superior loop and the second end is connected to the inferior loop. The construct of the compliance member and loops provides a first force and a second force, both forces resistant to movement of the spinal segment. The first force resists flexion of the spinal segment and the second force resists extension of the spinal segment. Such resistance occurs when the spinous process motion creates tensile or compressive forces or some combination thereof in the compliance member.

An additional compliance member can be added to the device described above. In this case, the second compliance member is coupled with the superior and inferior loops. The construct of the second compliance member and loops provides a first force and a second force. Both forces are resistant to movement of the spinal segment. The first force resists flexion and the second force resists extension. The first and second compliance members may be mechanically coupled to a cross-member to provide lateral stabilization. The second compliance member is located across the midline of the spinal segment. The second compliance member is positioned essentially parallel to the first compliance member. The compliance member may comprise a spring. The force resistant to flexion may occur when the compliance member is elongated and the force resistant to extension may occur when the compliance member is compressed. The force resistant to extension may occur when the spring or compliance member is at least partially compressed or fully compressed.

In another aspect of the present invention, a device for restricting movement of a spinal segment comprises a first tether having an upper and lower strap. The upper strap is disposed around a superior surface of a superior spinous process and the lower strap is disposed around an inferior surface of the superior spinous process. A compliance member has a first end and a second end opposite the first end. The first end is coupled with the first tether. A second tether has an upper and lower strap with the upper strap disposed around a superior surface of the inferior spinous process and the lower strap disposed around an inferior surface of the inferior spinous process. The second tether is coupled with the second end of the compliance member. The construct of the compliance member and straps provides a first force and a second force. Both forces are resistant to movement of the spinal segment. Specifically, the first force is resistant to flexion of the spinal segment and the second force is resistant to extension of the spinal segment. Changing the length or tension of the upper strap of the first tether and/or the length or tension of the lower strap of the second tether adjusts the resistance to flexion. Alternatively or simultaneously, changing the length or tension of the lower strap of the first tether and/or changing the length or tension of the upper strap of the second tether adjusts the resistance to extension.

Another aspect of the present invention includes a device for restricting movement of a spinal segment. The device comprises a first tether having an upper and lower strap. The upper strap is disposed around a superior surface of a superior spinous process and the lower strap is disposed around an inferior surface of the superior spinous process. A compliance member has a first end and a second end with the second end opposite the first end. The first end of the compliance member is coupled with the first tether. A second tether is coupled to the sacrum. The second tether is coupled with the second end of the compliance member. The construct of the compliance member, straps and tether provides a first force and a second force. Both forces are resistant to movement of the spinal segment. The first force is resistant to flexion of the spinal segment and the second force resistant to extension of the spinal segment.

Coupling the second tether to the sacrum can be accomplished in a number of ways. If a sacral spinous process is present, the second tether may have an upper and lower strap with the upper strap disposed around a superior surface of an inferior spinous process and the lower strap disposed around an inferior surface of the inferior spinous process. If a sacral spinous process is insufficient for such attachment the second tether may be passed through a hole through the sacral spinous process. Such a hole may be created in situ to facilitate such passage of the second tether. Alternatively, the second tether may attach to the sacrum through alternative means of fixed attachment, such as screws, bone anchors, hooks, dowels, staples, pins, sutures or the like. When attached to a sacrum, the second tether may be attached to an alar surface of the sacrum, typically with alar screws. Further details regarding sacral attachment can be found in U.S. application Ser. No. 11/827,980 (Attorney Docket No. 026398-000120US), filed on Jul. 13, 2007, the full disclosure of which is incorporated herein by reference.

Another aspect of the present invention includes a method for restricting movement of a spinal segment. The method comprises providing a constraining device that has a tether and a compliance member coupled together. The tether is coupled to a superior spinous process and an inferior spinous process or a sacrum so that the construct of the compliance member and tether provides a force resistant to flexion and extension of the spinal segment. Coupling the tether comprise encircling the superior spinous process or inferior spinous process with the tether. Coupling the tether may also include piercing the interspinous ligament and passing the tether through the perforation. The tether may be fixed to a target size.

Additionally, the device may be adjustable and may further comprise a second compliance member. The construct of the second compliance member and tether provides a force resistant to flexion of the spinal segment and a force resistant to extension of the spinal segment.

In another aspect of the present invention, a device for restricting the movement of a spinal segment comprises a dynamic constraint having a superior loop and an inferior loop. The superior loop captures a superior spinous process and the inferior loop captures an inferior spinous process. A compliance member has a first end and a second end. The second end is opposite the first end. The compliance member may comprise a torsion spring. The first end is connected to the superior loop and the second end is connected to the inferior loop. The construct of the compliance member and loops provides a force resistant to extension of the spinal segment.

Another aspect of the present invention includes a device for controlling extension of a spinal segment. The device comprises a constraint coupled to a superior spinous process and an inferior spinous process. The constraint includes a first strap coupled to the inferior surface of the superior spinous process and a second strap coupled to the superior surface of the inferior spinous process. Each strap has two ends. A first compliance member has a first end and a second end opposite the first end. The first end of the first compliance member is connected to the first end of the first strap and the second end of the first compliance member is connected to the first end of the second strap. A second compliance member has a first end and a second end opposite the first end. The second compliance member is located across the midline of the spinal segment. The second compliance member has a first end and a second end opposite the first end. The first end of the second compliance member is connected to the second end of the first strap and the second end of the second compliance member is connected to the second end of the second strap. The construct of the compliance members and straps provides a force resistant to extension of the spinal segment. Changing the length of the substantially inextensible straps adjusts the resistance to extension.

These and other embodiments are described in further detail in the following description related to the appended drawing figures.

### BRIEF DESCRIPTION OF THE DRAWINGS

FIG. 1A is a schematic diagram illustrating the lumbar region of a typical spine including the spinous processes (SP), facet joints (FJ), lamina (L), transverse processes (TP), and sacrum (S).

FIG. 1B is a schematic diagram illustrating a portion of the lumbar region of the spine taken along a sagittal plane.

FIG. 2 illustrates perspective view of a spinal implant of the type described in U.S. Patent Publication No. 2005/0216017 A1.

FIG. 3 is a schematic diagram illustrating a posterior view of a spinal segment with a constraint device attached thereto.

FIG. 4 is a schematic diagram illustrating an alternative embodiment of a constraint device.

FIG. 5 is a schematic diagram illustrating another embodiment of a constraint device.

FIG. 6A is a schematic diagram illustrating yet another embodiment of a constraint device.

FIG. 6B is a schematic diagram illustrating another embodiment of a constraint device with torsion spring(s) placed on either side of the interspinous ligament.

FIG. 6C is a schematic diagram illustrating another embodiment of a constraint device with torsion spring(s) placed between spinous processes.

FIG. 7 is a schematic diagram illustrating still another embodiment of a constraint device.

FIG. 8 is a schematic diagram illustrating another embodiment of a constraint device.

FIG. 9 is a schematic illustration of still another embodiment of a constraint device.

FIG. 10 is a schematic illustration of an exemplary method of deploying a spinous process constraint device including piercing the interspinous ligament.

FIG. 11 is a schematic diagram illustrating still another embodiment of a constraint device.

### DETAILED DESCRIPTION OF THE INVENTION

The following exemplary embodiments of methods and devices will be described in the context of applying a constraint around the spinous processes to restrict flexion and extension of a spinal segment. This is intended to be for illustrative purposes only and one of ordinary skill in the art will recognize that the methods and devices disclosed herein may be used in a number of other applications and therefore are not limited to spinal surgery. The features and advantages will become apparent upon reading the following detailed description and referring to the accompanying drawings in which like numbers refer to like parts throughout.

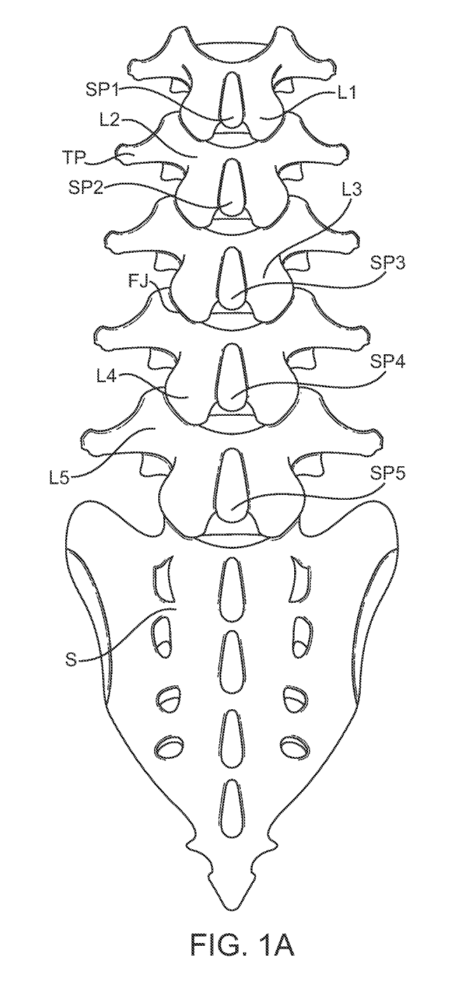

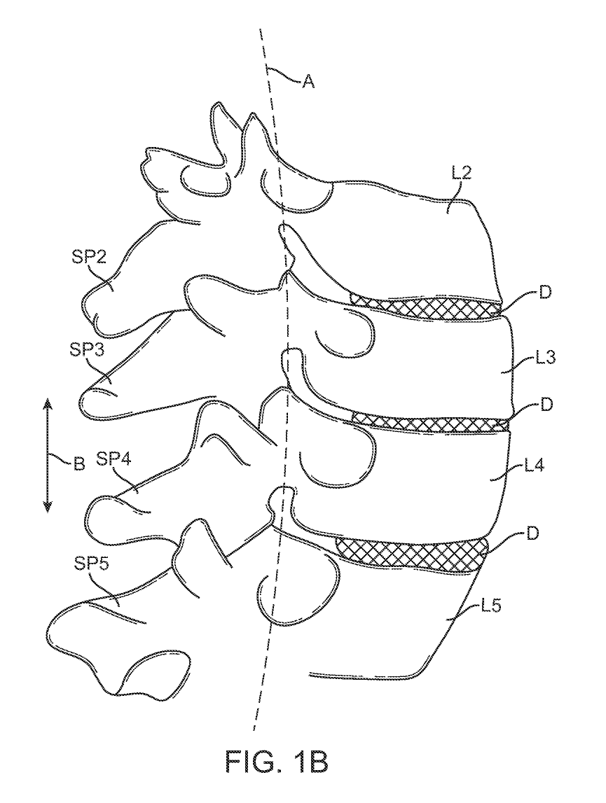

FIG. 1A is a schematic diagram illustrating the lumbar region of the spine including the spinous processes (SP), facet joints (FJ), lamina (L), transverse processes (TP), and sacrum (S). FIG. 1B is a schematic illustration showing a portion of the lumbar region of the spine taken along a sagittal plane and is useful for defining the terms “neutral position,” “flexion,” and “extension” that may be referred to in this disclosure.

As used herein, “neutral position” refers to the position in which the patient's spine rests in a relaxed standing position. The “neutral position” will vary from patient to patient. Usually, a neutral position will be characterized by a slight curvature or lordosis of the spine where the spine has a slight anterior convexity and slight posterior concavity. In some cases, the presence of the constraint of the present invention may modify the neutral position, e.g. the device may apply an initial force which defines a new neutral position having some flexion or extension of the untreated spine. As such, the use of the term “neutral position” is to be taken in context of the presence or absence of the device. As used herein, “neutral position of the spinal segment” refers to the position of a spinal segment when the spine is in the neutral position.

Furthermore, as used herein, “flexion” refers to the motion between adjacent vertebrae in a spinal segment as the patient bends forward. Referring to FIG. 1B, as a patient bends forward from the neutral position of the spine, i.e. to the right relative to a curved axis A, the distance between individual vertebrae L on the anterior side decreases so that the anterior portion of the intervertebral disks D are compressed. In contrast, the individual spinous processes SP on the posterior side move apart in the direction indicated by arrow B. Flexion thus refers to the relative movement between adjacent vertebrae as the patient bends forward from the neutral position illustrated in FIG. 1B.

Moreover, as used herein, “extension” refers to the motion of the individual vertebrae L as the patient bends backward and the spine extends from the neutral position illustrated in FIG. 1B. As the patient bends backward, the anterior ends of the individual vertebrae will move apart. The individual spinous processes SP on adjacent vertebrae will move closer together in a direction opposite to that indicated by arrow B.

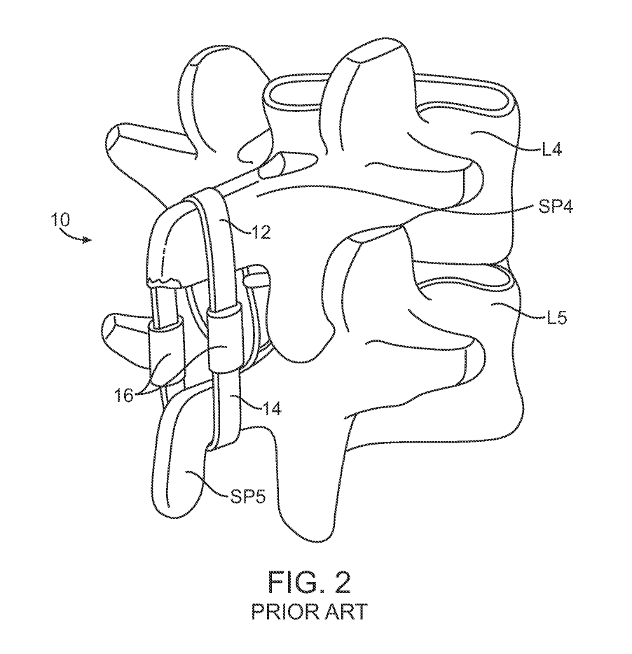

FIG. 2 shows a spinal implant of the type described in related U.S. Patent Publication No. 2005/02161017 A1, the contents of which are herein incorporated by reference. As illustrated in FIG. 2, an implant 10 typically comprises an upper strap component 12 and a lower strap component 14 joined by a pair of compliance members 16. The upper strap 12 is shown disposed over the top of the spinous process SP4 of L4 while the lower strap 14 is shown extending over the bottom of the spinous process SP5 of L5. The compliance member 16 will typically include an internal element, such as a spring or rubber block, which is attached to the straps 12 and 14 in such a way that the straps may be “elastically” or “compliantly” pulled apart as the spinous processes SP4 and SP5 move apart during flexion. In this way, the implant provides an elastic tension on the spinous processes which provides a force that resists flexion. The force increases as the processes move further apart. The straps themselves will be substantially inextensible so that the degree of elasticity or compliance may be controlled and provided solely by the compliance members 16. The phrase “substantially inextensible” as used herein, refers to any substance or material that does not considerably or significantly expand, extend, unfold, stretch, enlarge or swell. Other embodiments disclosed below will not only provide resistance to flexion of the spinal segment, but they will also provide resistance to extension of the spinal segment.

As used herein, “resistance” refers to an application of constraining force to resist motion between successive, usually adjacent, spinous processes such that increased motion of the spinous processes results in a greater constraining force. The resistance will, in the inventions described herein, inhibit motion of individual spinal segments during both flexion and extension or in extension alone. A constraining force is transmitted directly to the spinous processes or to one or more spinous process and the sacrum. Resistant forces can be supplied by springs, elastomeric elements, and other such devices, for example.

While FIG. 2 illustrates a single tether structure wrapped around adjacent spinous processes without an intermediate spinous process therebetween, one will appreciate that a spinal segment having more than two spinous processes may be tethered together using one or more tether structures.

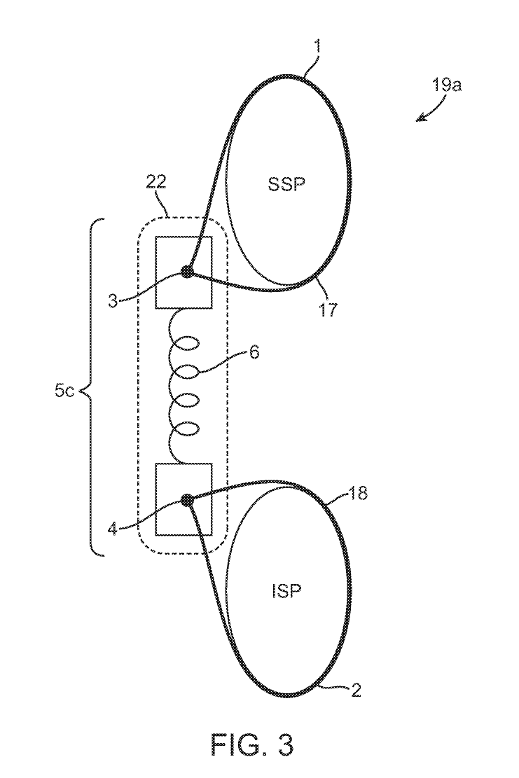

Referring now to FIG. 3, a device 19a according to the present invention for controlling the movement of a spinal segment will be described. FIG. 3 illustrates one exemplary embodiment of the present invention. This posterior view of the constraint implant device 19a includes a first strap 1 and a second strap 2, two fasteners (superior 3, inferior 4) and a single compliance member 5c that includes a spring 6. An optional sheath 22 may cover compliance; member 5c.

The first strap 1 will typically be a continuous band, cable, cord, or other structure flexibly adapted to capture a superior spinous process SSP as described in more detail in the related prior applications which have been incorporated herein by reference. The second strap 2 will typically comprise a band, cable, or the like which is constructed similarly if not identically to the first strap 1 to capture an inferior spinous process ISP. In other embodiments, the second strap 2 may be coupled to the sacrum instead of an inferior spinous process. Coupling may be achieved by fastening mechanisms such as screws or by threading the second strap 2 through a hole in the sacrum. The straps will usually be flexible but substantially inextensible so that they allow minimum elongation under a tensile load. A third strap 17 is coupled to (and may be contiguous with) the first strap 1 and passes below the superior spinous process SSP. A fourth strap 18 is coupled to (and may be contiguous with) the second strap 2 and passes above the inferior spinous process ISP. As appreciated by one skilled in the art, the straps (mentioned in this embodiment or any other embodiment of this invention) may be discrete straps or the straps may be contiguous with one another. Straps 1 and 2 resist forces when the spinal segment is in flexion and straps 17 and 18 resist forces when the spinal segment is in extension.

In general, the spring 6 or other elastomeric member of the compliance device will elastically elongate as tension is applied by the first and second straps 1 and 2. When the spinous processes or spinous process and sacrum move apart during flexion of the constrained spinal segment, the first and second straps 1 and 2 will also move apart and thus spring 6 will resist flexion. The spring 6 or elastomeric member will shorten when force is applied during extension. When the spinous processes or spinous process and sacrum move together during extension of the constrained spinal segment, the third strap coupled to the inferior surface of the superior spinous process and the fourth strap coupled to the superior surface of the inferior spinous process will also move together and therefore spring 6 will resist extension.

The compliance member 5c will elastically resist the spreading with a force determined by the mechanical properties of the spring 6 or other spring-like element. In particular, the spring 6 will be selected to have a tensile or elastic stiffness, also known as a spring constant, such that the construct of the compliance member and straps will provide adequate resistance to flexion and extension in a variety of patients. Such resistance occurs when the spinous process motion creates tensile or compressive forces or some combination thereof in the compliance member. The compliance member absorbs energy that would otherwise be transmitted into the tissues.

The compliance member 5c will include a fixed attachment component (i.e., fastener) 3 that couples the superior tether to the compliance member. A second fastener 4, attaches the inferior tether 2 to the compliance member 5c. Located between the fasteners, a helical spring or other spring-like structure 6 may be formed from a single piece of material or may comprise multiple components fastened together. Typically, the spring 6 and both tether fasteners 3 and 4 will be formed from a metal such as titanium, but optionally may be a polymer, ceramic, reinforced glass or other composite, or other material having desired elastic and mechanical properties and capable of being formed into the desired geometry.

Alternatively, a suitable polymeric material will be polyether ether ketone (PEEK). The fastener may comprise a housing having an aperture in which the tether is fed through. A lock having a roller and set screw create a friction fit between the tether and roller/housing, thereby locking the tether in position. Other embodiments of a locking mechanism simply involve a screw clamp or other fixture. Further details on fastening mechanisms are disclosed in U.S. Provisional Patent Application Nos. 61/059,538 (Attorney Docket No. 026398-000700US) and 61/059,543 (Attorney Docket No. 026398-000800US), the entire contents of which are incorporated herein by reference. Additional details on fastening are disclosed below and may be applied to any of the fasteners disclosed herein.

The exterior of compliance member 5c may be covered with a protective cover, such as the elastomeric sheath 22. The sheath may be placed over the body of the compliance member, as illustrated by dashed lines in FIG. 3, in order to prevent the intrusion of tissue and body materials into the spaces between the turns of the coil and interior of the compliance member 5c. The sheath 22 may also provide alignment support to the spring to prevent the spring from bowing, bending or otherwise diverging from an essentially axial alignment before, during and after movement including when the spinal segment undergoes flexion and extension.

Pre-tensioning or pre-loading the compliance member to a desired value is possible. One way to accomplish this is by changing the length of the constraining structure such that a small amount of tension is held by the constraining structure when the spine is in a neutral position. Alternatively, pre-loaded compliance elements can be provided to pre-load the constraining structure without changing its length. The tension or compression elements utilized in the compliance members of the present invention, such as springs, elastomeric bodies, and the like, will typically present little or no elastic resistance when they are first deformed. Thus, there will be some degree of elongation or compression of the compliance members prior to the spinal segment receiving a therapeutic resistance. To provide more immediate relief, the tension or compression members may be pre-loaded to have an initial static resistive force which must be overcome to initiate deformation. In this way, a constrained spinal segment will not begin to flex or extend at the instant the patient begins to flex or extend her or his spine which is an advantage when treating lax spinal segments. Pre-loading is further described in co-pending U.S. application Ser. No. 12/106,103 (Attorney Docket No. 026398-000410US), filed on Apr. 18, 2008, the full disclosure of which is incorporated herein by reference.

In alternative embodiments disclosed herein below, a second compliance member may be added to the structure shown in FIG. 3. The second compliance member may be located across the midline of the spinal segment from the first compliance member and will typically be constructed similarly if not identically to the first compliance member, although the second compliance member may also take a different form than the first compliance member.

One or both ends of the straps may be pre-attached to the fasteners on the compliance member(s). The straps of the two subassemblies can then be threaded through a perforation in the interspinous ligament, positioned to capture the respective spinous processes and secured. Various methods of deploying, fastening, and locking the straps including methods of how to adjust the length and tension can be found in co-pending application Ser. No. 11/875,674 (Attorney Docket No. 026398-000150US), for example.

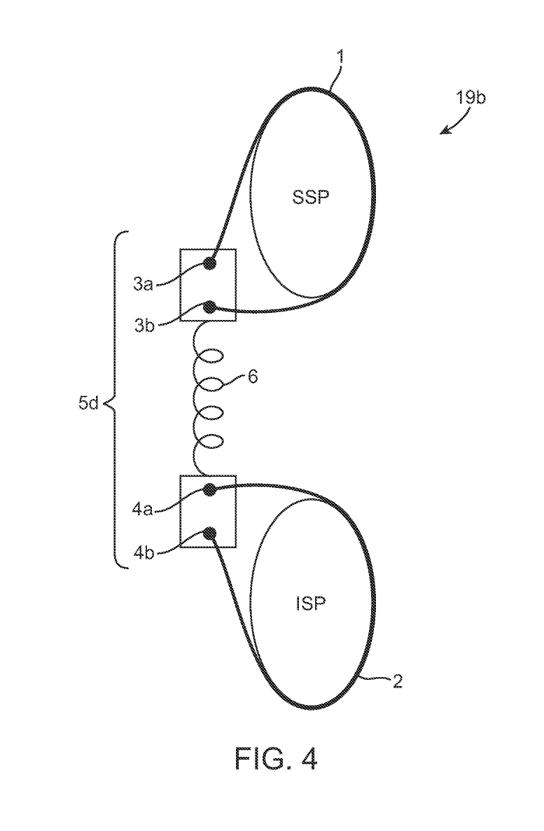

An alternative embodiment of the present invention is shown in FIG. 4. The constraint implant device 19b comprises a superior strap 1 and an inferior strap 2, four fasteners (upper superior 3a, lower superior 3b, upper inferior 4a, lower inferior 4b) and a single compliance member 5d that includes a spring 6. In this embodiment, each end of the 4 total ends of each of the two total straps attach at a different location on the compliance member (i.e. fasteners 3a, 3b, 4a, 4b).

The straps described in the embodiments above may be fastened to the a compliance member using a pin. The pin may be anchored in a pair of receiving holes, and a free end of the tether wrapped over the pin and firmly attached to itself with adhesive, suture, stitching, clamping etc. Usually, the fixed tether structure will be pre-attached at the time of manufacture so that the treating physician can implant each of the pair of compliance members, with one end of each tether structure attached to a fixed tether connector. The remaining free ends of each strap may then be deployed around the spinous processes (or attached to a sacrum) in any of the patterns disclosed herein or in a pattern further shown and described in co-pending application Ser. No. 11/875,674 (Attorney Docket No. 026398-000150US), the full disclosure of which is incorporated herein by reference.

Fasteners may also include locks, clamps, and other such mechanisms used to secure a strap. One such mechanism includes a housing, a roller element, and a locking member engaged with the housing. The housing has a central channel and a first side surface which defines an entry aperture and a second side surface which defines an exit aperture. A side channel extends between the entry and exit apertures. The roller element has a sidewall with an aperture. The roller element permits passage of the tether. The roller element winds the strap and creates a friction fit between the roller element, the housing and the strap. The locking member is engaged with the housing to prevent release of the roller from the housing. Fasteners can take the same form as those described in U.S. application Ser. No. 61/059,530 (Attorney Docket No. 026398-000600US), filed on Jun. 6, 2008, and U.S. application Ser. No. 61/059,538 (Attorney Docket No. 026398-000700US), filed on Jun. 6, 2008, the full disclosures of which are incorporated herein by reference. One of ordinary skill in the art will recognize that many other fasteners may be used such as a set screw, a locking clasp, spring loaded detents, etc, any of which may be substituted in any of the embodiments disclosed herein.

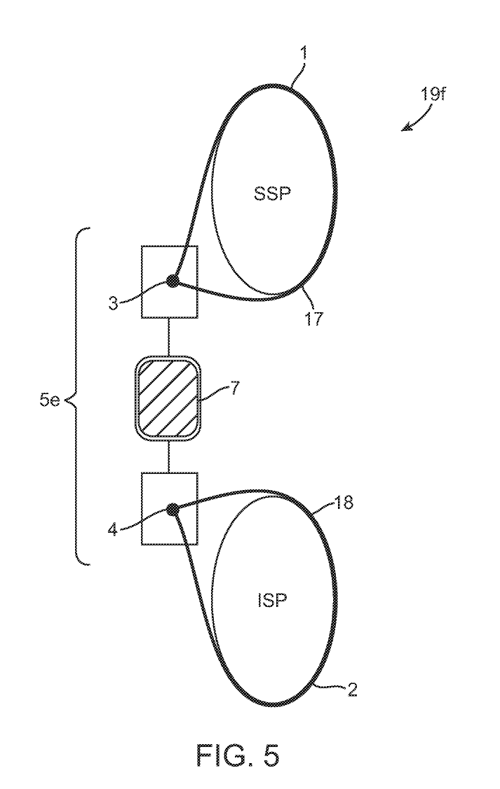

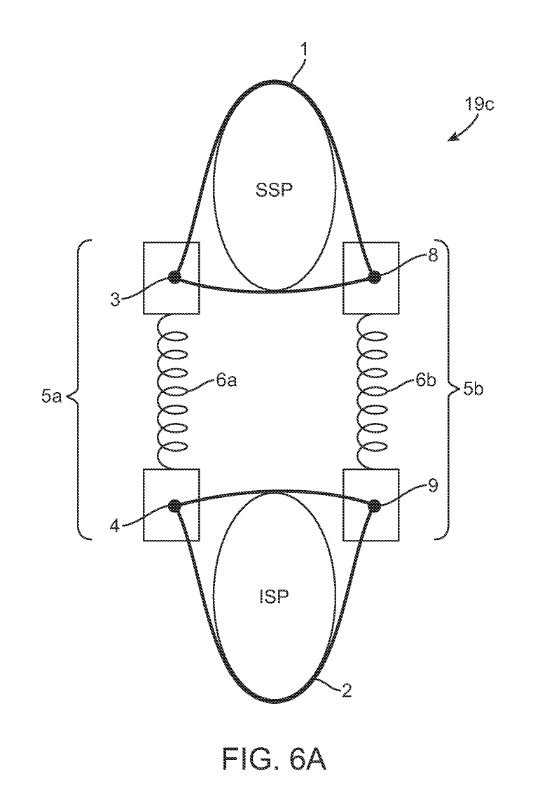

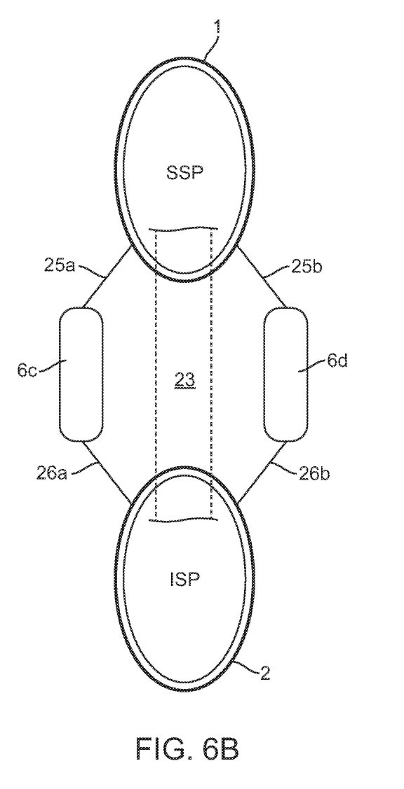

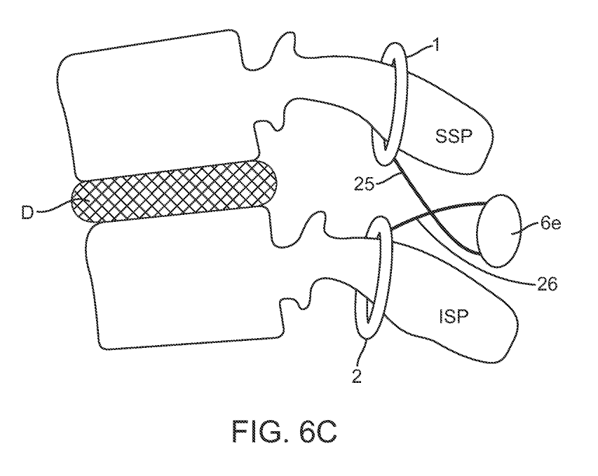

FIG. 5 is a schematic diagram illustrating an alternative embodiment of the constraint implant device 19f depicting a compliance member 5e that includes an elastomeric member 7 instead of a spring. The entire structure can be molded or cast from an elastomeric material having mechanical properties that provide the desired elastic stiffness or spring force, as set forth in co-pending U.S. application Ser. No. 12/106,103 (Attorney Docket No. 026398-000410US), filed on Apr. 18, 2008, the full disclosure of which has been previously incorporated herein by reference. A particularly suitable elastomer is silicone rubber, but other thermoplastics and thermosetting elastomers could also be used. It will be appreciated that the compliance member can include any type of component or material that produces the desired resistive force. A spring, elastomeric member and rubber bumper are only a few examples of many available options. If two compliance members are employed, as shown in FIG. 6A, for example, any combination of springs or elastomeric members can be used.

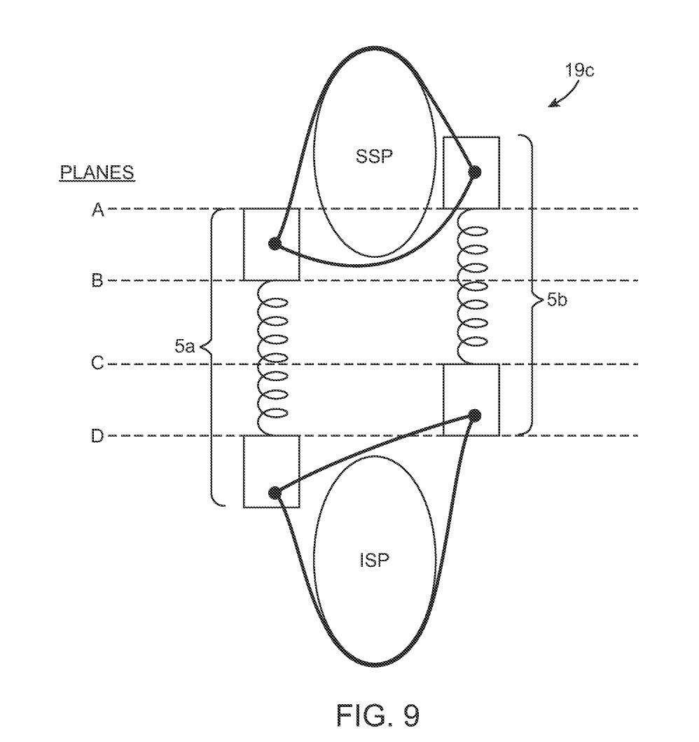

FIG. 6A is a schematic diagram illustrating another alternative embodiment of the constraint implant device 19c of the present invention comprising a superior strap 1 and an inferior strap 2, four fasteners 3, 4, 8, 9 and two compliance members 5a, 5b that each include a spring 6a, 6b. The superior strap 1 is coupled and attached to fasteners 3 and 8. The inferior strap 2 is coupled and attached to fasteners 4 and 9. In this embodiment, the straps are operatively coupled to each other via a spring to form a continuous integral loop. The two compliance members 5a, 5b are positioned across the spinal midline from one another. The second compliance 5b member can be essentially parallel to the first compliance member 5a. Alternatively, the compliance members 5a, 5b can be offset from one another. This can be seen in FIG. 9 by comparing the orientation planes A-D. For example, the inferior end of compliance member 5b resides between planes C and D while the inferior end of compliance member 5a resides outside plane D. Methods of deploying, fastening, and locking the straps including methods of how to adjust the tension using two compliance members are disclosed in this application and may be applied to this embodiment as well.

In another embodiment of the present invention shown in FIG. 6B a torsion spring has been substituted for a standard spring as a compliance member. The torsion spring(s) can be designed with two concurrent or concentric coils to enable a different resistive stiffness in extension and flexion. If preservation of the interspinous ligament (ISL) is desired, torsion springs 6c, 6d can be placed on either side of the interspinous ligament (ISL) 23. A first arm 25a of the torsion spring 6c is attached to the superior strap 1 and the second arm 26a of the torsion spring 6c is attached to the inferior strap 2. A first arm 25b of the torsion spring 6d is attached to the superior strap 1 and the second arm 26b of the torsion spring 6d is attached to the inferior strap 2. As the spine segment flexes or extends, the torsion springs are engaged so that they resist the divergence (in flexion) or convergence (in extension) of the spinous process.

Alternatively, as shown in FIG. 6C, the torsion spring 6e can be placed between the spinous process if the ISL is resected. Straps capture the superior and inferior spinous processes by encircling them. A first arm 25 of the torsion spring is attached to the superior strap 1 and the second arm 26 is attached to the inferior strap 2. As the spine segment flexes or extends, the torsion spring is engaged so that it resists the divergence (in flexion) or convergence (in extension) of the spinous process. The torsion spring may provide differential resistance to extension and flexion, such that its resistive force is greater in one motion than the other. Relative to some of the other embodiments of compliance elements discussed herein, a torsion spring is easier to design to provide such differential resistance to flexion and extension.

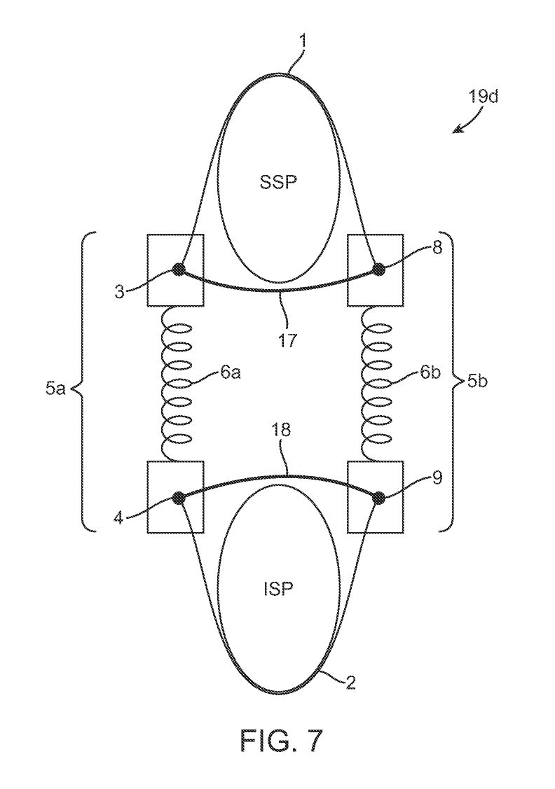

In yet another embodiment of the present invention, FIG. 7 illustrates a device 19d with a superior tether 1 and an inferior tether 2, two straps (17, 18) four fasteners 3, 4, 8, 9 and two compliance members 5a, 5b that each include a spring 6a, 6b. Elements 6a and 6b generally take the same form as previously described in FIG. 6A, for example. In this embodiment, the two tethers and two straps are distinct non contiguous strands. Accordingly, there are eight ends total to fasten to 4 different locations on the compliance member. One end of the superior tether 1 and one end of the first strap both attach to fastener 3. The opposite end of the superior tether 1 and the other end of the first strap both attach to faster 8. One end of the inferior tether 2 and one end of the second strap 18 both attach to fastener 4. The opposite end of the inferior tether 2 and the other end of the second strap 18 attach to faster 9.

In certain instances, however, the inferior tether structure 2 may comprise separate bands, cables, cords, or the like. The use of such separate tether structures for inferior attachment to the sacrum, for example, are described in more detail in co-pending application Ser. No. 11/827,980 (Attorney Docket No. 026398-000120US), the full disclosure of which has been previously incorporated herein by reference. In some patients, the geometry of another spinous process may be such that the strap may not be adequately secured by encircling the spinous process. For example, the spinous process may be defective or be intraoperatively damaged. In such instances, the methods may further comprise inserting a screw into the vertebral body and attaching the straps to the screw, for example. In still other instances, a screw may be inserted into a pedicle and the straps attached to the screw. Such techniques are further described in U.S. application Ser. No. 12/106,049 (Attorney Docket No. 026398-000151US), filed on Apr. 18, 2008, the full disclosures of which are incorporated herein by reference.

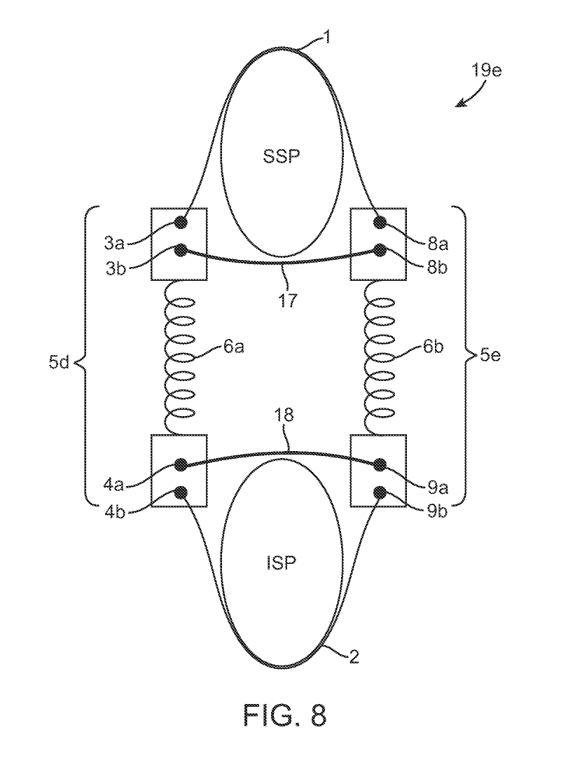

FIG. 8 shows an alternative embodiment of the device 19e comprising a superior tether 1 and an inferior tether 2, two straps 17, 18, eight fasteners 3a, 3b, 4a, 4b, 8a, 8b, 9a, 9b and two compliance members 5d, 5e that each include a spring 6a, 6b. The two total tethers 1, 2 and two total straps 17, 18 are distinct non contiguous strands similar to those shown in FIG. 7. However, in this embodiment, each end of the tethers and straps attach to a different location on the compliance member. Accordingly, there are eight ends total to fasten to eight locations on the compliance member. One end of the first strap 17 is attached at fastener 3b. The opposite end of the first strap 17 is attached at fastener 8b. One end of the second strap 18 is attached at fastener 4a. The opposite end of the first strap 18 is attached at fastener 9a. One end of the superior tether 1 is attached at fastener 3a. The opposite end of the superior tether 1 is attached at fastener 8a. One end of the inferior tether 2 is attached at fastener 4b. The opposite end of the inferior tether 2 is attached at fastener 9b. Any subset or combination of the aforementioned tether/strap ends can be pre-attached using pins, screws, rivets, adhesive, or the like. Other combinations of pre-attached and intraoperatively attached fasteners can also be employed. Such techniques are further described in U.S. application Ser. No. 12/106,103 (Attorney Docket No. 026398-000410US) filed on Apr. 18, 2008, the full disclosure incorporated herein by reference. Some advantages of this embodiment are that it allows simple and independent adjustment of the superior and inferior tethers/straps.

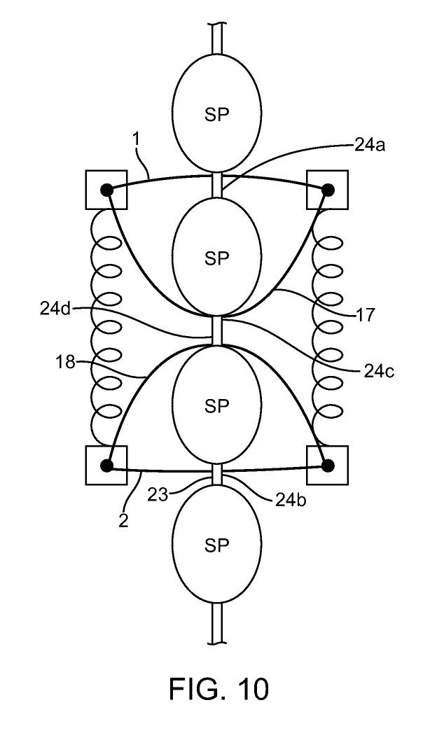

In order to position a constraint around spinous processes to restrict flexion and extension of a spinal segment, the interspinous ligament must first be pierced multiple times. An initial incision is made over the spinal area of interest. A piercing tool having a sharp distal end may be used to access and pierce the interspinous ligament while avoiding other anatomy. This surgical approach is desirable since it keeps the supraspinous ligament intact and minimizes damage to the multifidus muscle and tendons and other collateral ligaments. In addition to accessing and piercing the interspinous ligament, the piercing tool also advances or threads the tether through the perforation or facilitates advancement of the tether by threading a leader through the perforation. In this embodiment, the tether portion of a spinous process constraint device is releasably coupled directly with the piercing tool. FIG. 10 shows the most relevant anatomy in order to more clearly illustrate piercing an interspinous ligament 23 to form a first perforation 24a above a superior spinous process and advancing a first end of a first tether 1 through the first perforation. Piercing the interspinous ligament below an inferior spinous process forms a second perforation 24b so that a second end of a second tether 2 may be advanced through the second perforation. The first and second tethers are joined to a compliance member to form an extensible tether structure wherein the structure couples the superior and the inferior spinous processes together while still permitting extension therebetween. The tethers can be adjusted to set a relative distance or angle between the upper and lower spinous processes to a target value.

The elastic resistance to flexion applied by the constraining structure of the present invention is exerted when the spinal segment moves beyond a neutral position and will depend on several factors including the elastic characteristics of the constraining structure, the position of the constraining structure on the spinous processes, the dimensions of the constraining structure, and the patient's anatomy and movement. The constraining structure will usually be positioned so that the upper and lower tethers engage the middle anterior region of the spinous process, and the mechanism of the constraining structure will usually be adjusted so that the tethers are taut (e.g. free from slack) when the spinal segment is in a neutral position. As the segment flexes beyond a neutral position, the constraining structure will immediately provide an elastic resistance.

The present invention is also particularly concerned with constraining adjacent spinous processes to restrict extension of a spinal segment. This further comprises piercing an interspinous ligament below a superior spinous process to form perforation 24c and advancing a first end of a first strap 17 through the penetration as shown in FIG. 10. Piercing the interspinous ligament above an inferior spinous process forms a separate perforation 24d so that a first end of a second strap 18 may be advanced through the penetration. The first and second straps are joined to a compliance member to form a substantially inextensible structure wherein the structure couples the upper and the lower spinous processes together to restrict extension therebetween.

Piercing tools, capture elements and methods used to pierce the interspinous ligament and thread tethers through perforations can be found, for example, in U.S. application Ser. No. 61/059,530 (Attorney Docket No. 026398-000600US), filed on Jun. 6, 2008, and U.S. application Ser. No. 12/106,049 (Attorney Docket No. 026398-000151US), filed on Apr. 18, 2008, the full disclosures of which are incorporated herein by reference.

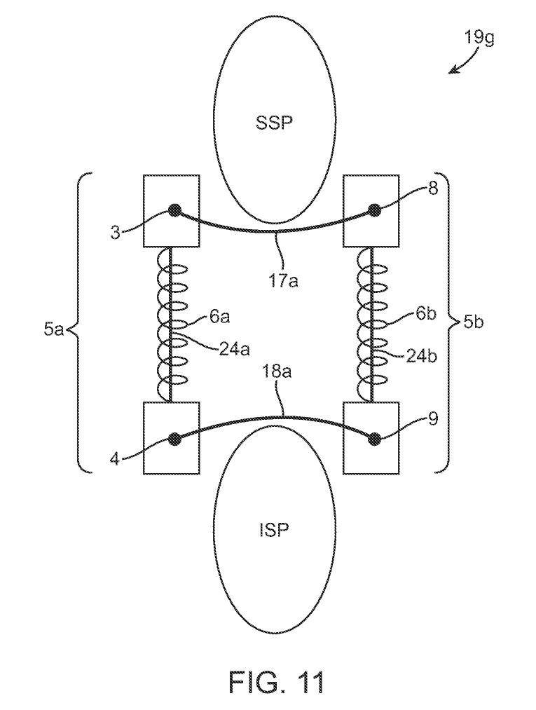

FIG. 11 shows an alternative embodiment of the present invention. Device 19g depicts four fasteners 3, 4, 8, 9 and two compliance members 5a, 5b, two stabilizer rods 24a, 24b and two straps 17a, 18a. In this embodiment, one end of the first strap 17a attaches to fastener 3. The opposite end of the first strap attaches to faster 8. One end of the second strap 18a attaches to fastener 4. The opposite end of the second strap 18a attaches to faster 9. The first strap 17a is coupled to the inferior surface of the superior spinous process SSP and a second strap 18a is coupled to the superior surface of the inferior spinous process ISP. A first compliance member 5a has a first end with a first fastener 3 and a second end, opposite the first end, with a second fastener 4. The first end of the compliance member is fastened to the first end of the first strap 17a with fastener 3. The second end is of the compliance member is fastened to the first end of the second strap 18a with fastener 4. The second compliance member 5b is located across a midline of the spinal segment. The first end of the compliance member is connected to the second end of the first strap 17a at fastener 8. The second end is connected to the second end of the second strap 18a at fastener 9. The first and second compliance members provide resistance to extension of the spinal segment. Changing the length of the first or second straps adjusts the resistance to extension. For example, a compliance member could enable a range of magnitudes of extension resistance from a minimum value of no resistance when the straps are extremely loose to a maximum resistance value that depends on the distance between the superior and inferior attachment points of the straps to the compliance members. In treating stenosis, for example, the surgeon could shorten the effective length of the interspinous straps until the foramen begins to open or until the surgeon otherwise believes that the extension resistance is therapeutically sufficient.

Stabilizer rods 24a and 24b, as shown in FIG. 11, may also be positioned inside the springs 6a and 6b, respectively, to provide alignment support to the spring to prevent the spring from bowing, bending or otherwise diverging from an essentially axial alignment before, during and after movement including when the spinal segment undergoes flexion and extension. Axial alignment stabilizers can be rods, tubes, or other such devices, for example. The stabilizer rods can optionally be used in any other embodiments of the present invention where a spring is used with a compliance device.

Lateral stabilization can be accomplished by the use of non extension-limiting cross-members, for example as disclosed in U.S. application Ser. No. 11/777,366 (Attorney Docket No. 026398-000110US), filed on Jul. 13 2007, the full disclosure of which is incorporated herein by reference. These cross-members could connect compliance members across the midline providing a force that would resist outward bending or bowing of the compliance members. Alternatively, single or multiple element components could be added to the described constructs to resist bucking or bulging.

Additional embodiments are envisioned where the straps/tethers couple to other vertebral processes such as pedicles, lamina, and/or transverse processes. It may be desirable to couple to these structures if a spinous process is either not present or incompetent, for example due to osteoporosis or surgical interventions such as a wide laminectomy. Coupling to other vertebral processes that are located more laterally or more anteriorly may additionally permit the device to restrict motions such as lateral bending or axial rotation in addition to flexion and extension. Furthermore, coupling to or bridging multiple motion segments may permit restriction of additional modes of motion such as vertebral translation or listhesis. Any of the constructs may be easily adjusted via percutaneous, minimally invasive means or via transcutaneous, non-invasive means as found in PCT Application No. PCT/US2007/081822 (Attorney Docket No. 026398-000140PC) filed on Oct. 18, 2007, the full disclosure of which is incorporated herein by reference, for example.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A device for controlling the movement of a spinal segment, said device comprising:
a constraint coupled to a superior spinous process and an inferior spinous process, wherein the constraint comprises a first strap configured to capture the superior surface of the superior spinous process, and a second strap configured to capture the inferior surface of the inferior spinous process;
a third strap coupled to the first strap and configured to capture the inferior surface of the superior spinous process;
a fourth strap coupled to the second strap and configured to capture the superior surface of the inferior spinous process; and
a first compliance member having a first end and a second end opposite the first end, wherein the first end is connected to the first and third straps and the second end is connected to the second and fourth straps, wherein the construct of the compliance member and straps provides resistance to extension and flexion of the spinal segment.

2. A device for controlling the movement of a spinal segment, said device comprising:
a first strap having a first end and a second end, wherein the first strap captures a superior spinous process;
a second strap having a first end and a second end, wherein the second strap captures an inferior spinous process,
wherein the first and second straps are substantially inextensible; and
a first compliance member, having a first end connected to at least one of the first and second ends of the first strap and a second end opposite the first end connected to at least one of the first and second ends of the second strap,
wherein the construct of the first compliance member and straps provides resistance to extension and flexion of the spinal segment.

3. The device of claim 2, further comprising a second compliance member, having a first end and a second end opposite the first end, positioned across a spinal segment midline from the first compliance member,
wherein the first strap is coupled with the first end of the second compliance member and the second strap is coupled with the second end of the second compliance member.

4. The device of claim 3, wherein the second compliance member is positioned essentially parallel to the first compliance member.

5. The device of claim 2, wherein the compliance member comprises a spring.

6. The device of claim 2, wherein the device has a mechanism that is adjustable to allow tightening around the spinous processes.

7. The device of claim 2, wherein the straps are chosen from the group consisting of a leash, harness, lead, braid, plait, cord, belt, weave, strip, tie, and band.

8. The device of claim 2, wherein the natural anatomy of the interspinous ligament remains substantially intact after implantation of the device.

9. A method for restricting movement of a spinal segment, said method comprising:
implanting a constraint, said constraint having a first adjustable strap and second adjustable strap, each strap coupled to at least one compliance member;
capturing a superior spinous process with the first adjustable strap;
capturing an inferior spinous process with the second adjustable strap;
adjusting the straps, wherein the compliance member and straps provide a force resistant to extension and flexion of the spinal segment.

10. The method of claim 9, wherein capturing the spinous process with the straps does not require removal of the interspinous ligament.

11. The method of claim 9, wherein the first adjustable strap forms a first loop and the superior spinous process is captured therewith, the first loop comprising a superior and an inferior segment, and
wherein the second adjustable strap forms a second loop and the inferior spinous process is captured therewith, the second loop comprising a superior and an inferior segment.

12. The method of claim 9, wherein capturing comprises encircling the superior spinous process with the first strap.

13. The method of claim 9, wherein capturing comprises encircling the inferior spinous process with the second strap.

14. A method of claim 9, wherein adjusting the straps comprises adjusting the length of the straps.

15. A method of claim 9, wherein adjusting the straps comprises adjusting the tension in the straps.

16. A device for restricting movement of a spinal segment, said device comprising:
a constraint having a superior loop and an inferior loop, wherein the superior loop captures a superior spinous process and the inferior loop captures an inferior spinous process; and
a first compliance member having first and second ends, the second end opposite the first end, the first end connected to the superior loop and the second end connected to the inferior loop,
wherein the construct of the compliance member and the loops provides a first force and a second force, both forces resistant to movement of the spinal segment, the first force resistant to flexion thereof and the second force resistant to extension thereof.

17. The device of claim 16, further comprising a second compliance member coupled with the superior and inferior loops,
wherein the construct of the compliance members and loops provides a first force and a second force, both forces resistant to movement of the spinal segment, the first force resistant to flexion thereof and the second force resistant to extension thereof.

18. The device of claim 17, wherein the compliance members are mechanically coupled to a cross-member so as to provide lateral stabilization thereto.

19. The device of claim 17, wherein the second compliance member is located across a midline of the spinal segment.

20. The device of claim 17, wherein the second compliance member is positioned essentially parallel to the first compliance member.

21. The device of claim 16, wherein the first compliance member comprises a spring.

22. The device of claim 16, wherein the force resistant to flexion occurs when the first compliance member is elongated.

23. The device of claim 16, wherein the force resistant to extension occurs when the first compliance member is compressed.

24. The device of claim 16, wherein the force resistant to extension occurs when the first compliance member is fully compressed.

25. The device of claim 16, wherein the force resistant to extension occurs when the first compliance member is at least partially compressed.

26. The device of claim 16, wherein the superior and inferior loops are substantially inextensible.

27. The device of claim 16, wherein the superior and inferior loops comprise a fabric.

28. The device of claim 16, wherein the constraint is chosen from the group consisting of a leash, harness, lead, braid, plait, cord, belt, weave, strip, tie, and band.

29. The device of claim 16, wherein at least one spinous process is disposed between the superior spinous process and the inferior spinous process.

30. The device of claim 16, further comprising a first fastener coupled to the superior loop.

31. The device of claim 16, further comprising a second fastener coupled to the inferior loop.

32. A device for restricting movement of a spinal segment, said device comprising:
a first tether having an upper and lower strap, wherein the upper strap is disposed around a superior surface of a superior spinous process and the lower strap is disposed around an inferior surface of the superior spinous process;
a compliance member having a first end and a second end, the second end opposite the first end, wherein the first end is coupled with the first tether; and
a second tether having an upper and lower strap, wherein the upper strap is disposed around a superior surface of an inferior spinous process and the lower strap is disposed around an inferior surface of the inferior spinous process,
wherein the second tether is coupled with the second end of the compliance member, and
wherein the construct of the compliance member and tethers provides a first force and a second force, both forces resistant to movement of the spinal segment, the first force resistant to flexion thereof and the second force resistant to extension thereof.

33. The device of claim 32, wherein changing the length of the upper strap of the first tether or changing the length of the lower strap of the second tether adjusts the resistance to flexion.

34. The device of claim 32, wherein changing the stiffness of the compliance member changes the resistance to flexion or extension.

35. The device of claim 32, wherein changing the length of the lower strap of the first tether or changing the length of the upper strap of the second tether adjusts the resistance to extension.

36. The device of claim 32, wherein changing the tension of the upper strap of the first tether or changing the tension of the lower strap of the second tether adjusts the resistance to flexion.

37. The device of claim 32, wherein changing the tension of the lower strap of the first tether or changing the tension of the upper strap of the second tether adjusts the resistance to extension.

38. The device of claim 32, further comprising a second compliance member having a first end and a second end, the second end opposite the first end, wherein the first end is coupled with the first tether and the second end is coupled with the second tether, and
wherein the construct of the compliance members and tethers provides a first force and a second force, both resistant to movement of the spinal segment, the first force resistant to flexion thereof and the second force resistant to extension thereof.

39. The device of claim 38, wherein the second compliance member is located across the midline of the spinal segment.

40. The device of claim 38, wherein the second compliance member is positioned essentially parallel to the first compliance member.

41. The device of claim 32, wherein the compliance member comprises a spring.

42. The device of claim 32, wherein the force resistant to flexion occurs when compliance member is elongated.

43. The device of claim 32, wherein the force resistant to extension occurs when the compliance member is compressed.

44. The device of claim 32, wherein the force resistant to extension occurs when the compliance member is fully compressed.

45. The device of claim 32, wherein the first and second tethers are substantially inextensible.

46. The device of claim 32, wherein one of the tethers comprises a section that is fabric.

47. The device of claim 32, wherein one of the tethers is chosen from the group consisting of a leash, harness, lead, braid, plait, cord, belt, strip, tie, weave, and band.

48. The device of claim 32, wherein at least one spinous process is disposed between the superior spinous process and the inferior spinous process.

49. A device for restricting movement of a spinal segment, said device comprising:
a first tether having an upper and lower strap, wherein the upper strap is disposed around a superior surface of a superior spinous process and the lower strap is disposed around an inferior surface of the superior spinous process;
a compliance member having a first end and a second end, wherein the first end is coupled with the first tether; and
a second tether coupled to the sacrum,
wherein the second tether is coupled with the second end of the compliance member, and
wherein the construct of the compliance member, straps and tethers provides a first force and a second force, both resistant to movement of the spinal segment, the first force resistant to flexion thereof and the second force resistant to extension thereof.

50. A method for restricting movement of a spinal segment, said method comprising:
providing a constraining device having a tether and a compliance member coupled therewith; and
coupling the tether to a superior spinous process and an inferior spinous process or a sacrum so that the construct of the compliance member and tether provides a force resistant to flexion of the spinal segment and a force resistant to extension thereof.

51. The method of claim 50, wherein coupling the tether comprises encircling the superior spinous process or inferior spinous process with the tether.

52. The method of claim 50, wherein the constraining device further comprises a second compliance member, wherein the construct of the second compliance member and tether provides a force resistant to flexion of the spinal segment and a force resistant to extension thereof.

53. The method of claim 50, further comprising the step of adjusting length or tension in the constraining device.

54. The method of claim 50, wherein coupling the tether structure comprises piercing an interspinous ligament and passing the tether therethrough.

55. The method of claim 50, wherein the method further comprises fastening the tether into a target size.

56. A device for restricting movement of a spinal segment, said device comprising:
a dynamic constraint having a superior loop and an inferior loop, wherein the superior loop captures a superior spinous process and the inferior loop captures an inferior spinous process; and
a compliance member having first and second ends, the second end opposite the first end, the first end connected to the superior loop and the second end connected to the inferior loop;
wherein the construct of the compliance member and loops provides a force resistant to extension of the spinal segment

57. The device of claim 56, wherein the superior loop or the inferior loop encircles a spinous process.

58. The device of claim 56, wherein the compliance member comprises a torsion spring.

59. A device for controlling extension of a spinal segment, said device comprising:
a constraint coupled to a superior spinous process and an inferior spinous process;
a first strap coupled to the inferior surface of the superior spinous process;
a second strap coupled to the superior surface of the inferior spinous process;
a first compliance member having a first end and a second end opposite the first end, wherein the first end is connected to the first end of the first strap and the second end is connected to the first end of the second strap;
a second compliance member located across the midline of the spinal segment having a first end and a second end opposite the first end, wherein the first end is connected to the second end of the first strap and the second end is connected to the second end of the second strap, and
wherein the construct of the compliance members and straps provides a force resistant to extension of the spinal segment.
